(19) United States
(12) Patent Application Publication
(10) Pub. No.: US 2026/0174571 A1
(43) Pub. Date: Jun. 25, 2026

(54) DIAGNOSTIC METAMATERIAL CARDIOVASCULAR STENT

(71) Applicant: UNIVERSITY OF PITTSBURGH-OF THE COMMONWEALTH SYSTEM OF HIGHER EDUCATION, Pittsburgh, PA (US)

(72) Inventor: Amir Alavi, Pittsburgh, PA (US)

(21) Appl. No.: 19/125,090

(22) PCT Filed: Oct. 27, 2023
(86) PCT No.: PCT/US2023/036122

§ 371 Date: Apr. 28, 2025

Related U.S. Application Data
(60) Provisional application No. 63/420,309, filed on Oct. 28, 2022.

(51) Int. Cl. A61F 2/91; A61F 2/82; A61F 2/89; A61F 2/90; A61F 2/915; A61F 2/958

(52) U.S. Cl. CPC A61F 2/91; A61F 2/82; A61F 2/89; A61F 2/90; A61F 2/915; A61F 2/958; A61F 2230/0013; A61F 2250/0036

(57) ABSTRACT

A cardiovascular stent comprising: an active mechanical metamaterial, comprising: first and second electrically conductive components disposed relative to each other to act as opposite electrodes to induce contact electrification; wherein the first and second electrically conductive components, along with a dielectric component serving as a skeleton of the active mechanical metamaterial, form a lattice of snapping curved semicircular-shaped segments, wherein each of the snapping curved semicircular-shaped segments has an elastic snap-through instability mechanism; and wherein the lattice comprises periodic repeatable parallel rows of the snapping curved semicircular-shaped segments.

### RELATED APPLICATION

This application claims priority benefit under 35 U.S.C. § 119(e) of U.S. Provisional Application No. 63/420,309 filed Oct. 28, 2022, the contents of which are herein incorporated by reference.

### FIELD OF THE DISCLOSURE

### Technical Field

The present disclosure generally relates to the field of cardiovascular stents and mechanical metamaterials and methods for making and using the same.

### Background

Monitoring in-stent restenosis (ISR) is pivotal to target therapy and prognosis in patients with atherosclerosis. Stent implantation is a gold standard treatment for patients with atherosclerosis. Current technology is not suitable for accurately and reliably determining ISR in coronary arteries. Most wireless stents are equipped with sensor chips on their surface. Presence of any object on the outer surface of a cardiac stent is clinically unacceptable because it can obstruct the blood flow and injuring the blood vessel during insertion. In this disclosure, a new metamaterial-inspired stenting technology is presented for accurate and minimally invasive monitoring of the restenosis through the implanted stent without obstructing the blood flow. With cardiovascular disease (CVD) a major concern and more procedures of percutaneous coronary intervention (PCI) with a stent deployment being carried out yearly, development of such multifunctional wireless metamaterial stents that offer self-sensing and self-powering functionalities in addition to reliable mechanical properties is critical.

The present disclosure describes preferred smart metamaterial cardiovascular stents with diagnostic capabilities. The preferred metamaterial cardiovascular stents serve as stand-alone sensing media with wireless interrogation functionality for the evaluation of ISR and occlusion in coronary arteries in patients with atherosclerosis. The stenting technology of the present disclosure preferably facilitates early recognition of post-operative stent complications that would require additional intervention. The preferred smart stents of the present disclosure can continuously monitor restenosis via direct tracking of the changes of the radial compressive forces developed inside the stent structure. Since the smart stents of the present disclosure only rely on their constituent components for sensing the radial forces, they do not require any additional electronics, external power source, or data loggers for operation.

### BRIEF SUMMARY OF THE DISCLOSURE

In a preferred aspect, the present disclosure comprises a cardiovascular stent comprising: an active mechanical metamaterial (MM), comprising: first and second electrically conductive components disposed relative to each other to act as opposite electrodes to induce contact electrification; wherein the first and second electrically conductive components, along with a dielectric component serving as a skeleton of the active mechanical metamaterial, form a lattice of snapping curved semicircular-shaped segments, wherein each of the snapping curved semicircular-shaped segments has an elastic snap-through instability mechanism; and wherein the lattice comprises periodic repeatable parallel rows of the snapping curved semicircular-shaped segments.

In another preferred aspect of a cardiovascular stent of the present disclosure, one of the first and second electrically conductive components comprises a piezoelectric material that serves as a transducer to generate an electrical signal under an arterial pulsation.

In yet another preferred aspect of a cardiovascular stent of the present disclosure, a signal produced by the cardiovascular stent can be detected by electrodes placed on a user's skin and recorded by an electrocardiograph.

In another preferred aspect of a cardiovascular stent of the present disclosure, each of the electrically conductive components and the dielectric component comprises one or more materials selected from the group of polyurethane, polydimethylsiloxane, stainless steel, platinum-chromium, cobalt-chromium alloys, gold, titanium, polylactic acid and polyetheretherketone.

In a further preferred aspect of a cardiovascular stent of the present disclosure, a first one of the electrically conductive components comprises one or more materials selected from the group of polyurethane and polydimethylsiloxane; and wherein each of a second one of the electrically conductive components and the dielectric component comprises one or more materials selected from the group of stainless steel, platinum-chromium, cobalt-chromium alloys, gold, titanium, polylactic acid and polyetheretherketone.

In another preferred aspect of a cardiovascular stent of the present disclosure, one or more of the electrically conductive components and the dielectric component comprises one or more materials selected from the group of lead zirconate titanate, polyvinylidene fluoride or polyvinylidene difluoride.

In yet another preferred aspect of a cardiovascular stent of the present disclosure, one or more of the electrically conductive components and the dielectric component comprises one or more materials composed of auxetic microstructures.

In an additional preferred aspect of a cardiovascular stent of the present disclosure, the first and second electrically conductive components are embedded in the dielectric component.

In another preferred aspect of a cardiovascular stent of the present disclosure, a structure of the active mechanical metamaterial forms a composite matrix of the electrically conductive and dielectric components in a periodic manner.

In a further preferred aspect of a cardiovascular stent of the present disclosure, each of the snapping curved semicircular-shaped segments comprises a portion of each of the first electrically conductive component, the second electrically conductive component and the dielectric component.

In another preferred aspect of a cardiovascular stent of the present disclosure, the lattice comprises a 5 by 5 array of the snapping curved semicircular-shaped segments.

In another preferred aspect, the present disclosure comprises a cardiovascular stent comprising: an active mechanical metamaterial, comprising: first and second electrically conductive components disposed relative to each other to act as opposite electrodes to induce contact electrification; wherein the first and second electrically conductive components, along with a dielectric component serving as a skeleton of the active mechanical metamaterial, form a lattice of snapping curved semicircular-shaped segments, wherein each of the snapping curved semicircular-shaped segments has an elastic snap-through instability mechanism; wherein the lattice comprises periodic repeatable parallel rows of the snapping curved semicircular-shaped segments; and wherein one of the first and second electrically conductive components comprises a piezoelectric material that serves as a transducer to generate an electrical signal under an arterial pulsation.

In yet another preferred aspect, the present disclosure comprises a cardiovascular stent comprising: an active mechanical metamaterial, comprising: first and second electrically conductive components disposed relative to each other to act as opposite electrodes to induce contact electrification; wherein the first and second electrically conductive components, along with a dielectric component serving as a skeleton of the active mechanical metamaterial, form a lattice of snapping curved semicircular-shaped segments, wherein each of the snapping curved semicircular-shaped segments has an elastic snap-through instability mechanism; wherein the lattice comprises periodic repeatable parallel rows of the snapping curved semicircular-shaped segments; and wherein a first one of the electrically conductive components comprises one or more materials selected from the group of polyurethane and polydimethylsiloxane; and wherein each of a second one of the electrically conductive components and the dielectric component comprises one or more materials selected from the group of stainless steel, platinum-chromium, cobalt-chromium alloys, gold, titanium, polylactic acid and polyetheretherketone.

### BRIEF DESCRIPTION OF THE DRAWINGS

For the present disclosure to be easily understood and readily practiced, the present disclosure will now be described for purposes of illustration and not limitation in connection with the following figures, wherein:

FIG. 1A is a schematic of a smart, stand-alone metamaterial cardiovascular stent of the present disclosure with wireless integration functionality for continuous monitoring of local hemodynamic changes upon a re-narrowing condition;

FIG. 1B shows the signals of the manifest as tall waves in one of the chest (precordial) leads. These signals of preferred stents of the present disclosure correspond to various luminal re-narrowing rates and can thus be used for accurate ISR assessments;

FIG. 2A is a schematic of a preferred stent 110 of the present disclosure with a balloon-expandable expansion mechanism;

FIG. 2B is a schematic of a preferred stent 110 of the present disclosure and its functions;

FIG. 3 shows visions of the proposed multifunctional MM concept of the present disclosure for active sensing and energy harvesting: FIG. 3 shows in (a) a preferred composition of a “self-aware composite mechanical metamaterial” (SCMM) system of the present disclosure; FIG. 3 shows in (b) a preferred flying wing aircraft with self-diagnostic and energy harvesting wings made of a network of SCMM structures; and FIG. 3 shows in (c) a preferred self-powered and self-sensing cardiovascular stent using a preferred SCMM of the present disclosure for continuous monitoring of the artery radial pressure changes due to tissue overgrowth;

FIG. 4 shows power density for different available energy harvesting modalities;

FIG. 5A shows a segment of the conductive layers of a preferred 2D MM with parallel semicircular-shaped snapping segments of the present disclosure;

FIG. 5B shows two conductive layers created as 5 periodic repeatable segments of a preferred 2D MM with parallel semicircular-shaped snapping segments of the present disclosure;

FIG. 5C shows aligned conductive layers of a preferred 2D MM with parallel semicircular-shaped snapping segments of the present disclosure;

FIG. 5D shows the geometry of a preferred unit cell composed of the conductive layers and dielectric layers that are involved in the contact-separation process of a preferred 2D MM with parallel semicircular-shaped snapping segments of the present disclosure;

FIG. 5E and FIG. 5F show representations of the entire composite matrix of a preferred SCCM of the present disclosure composed of two conductive layers and dielectric layers in a periodic manner in a preferred 2D MM with parallel semicircular-shaped snapping segments of the present disclosure;

FIG. 5G shows a preferred manner of 3D printing a preferred SCCM of the present disclosure;

FIG. 6A shows a preferred self-sensing and self-charging 2D SCMM of the present disclosure with a 5×5 array of unit cells 20;

FIG. 6B shows a preferred SCCM of the present disclosure in the compacted state;

FIG. 6C shows preferred snapping mechanisms of elastic bulking semicircular-shaped snapping shells of the present disclosure;

FIG. 6D shows applied cyclic loads and the corresponding voltage generated by a preferred SCCM of the present disclosure.

### DETAILED DESCRIPTION OF PREFERRED EMBODIMENTS

In the following detailed description, reference is made to the accompanying examples and figures that form a part hereof, and in which is shown, by way of illustration, specific embodiments in which the inventive subject matter may be practiced. These embodiments are described in sufficient detail to enable those skilled in the art to practice them, and it is to be understood that other embodiments may be utilized and that structural or logical changes may be made without departing from the scope of the inventive subject matter. Such embodiments of the inventive subject matter may be referred to, individually and/or collectively, herein by the term “disclosure” merely for convenience and without intending to voluntarily limit the scope of this application to any single inventive concept if more than one is in fact disclosed.

The following description is, therefore, not to be taken in a limited sense, and the scope of the inventive subject matter is defined by the appended claims and their equivalents.

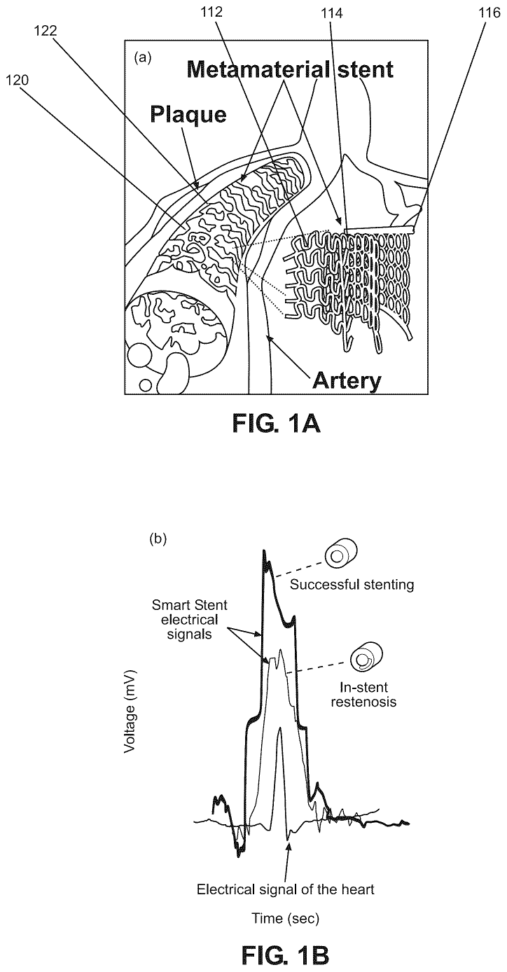

The smart metamaterial cardiac stents 110 of the present disclosure can self-monitor their condition. FIG. 1A shows a metamaterial cardiovascular smart stent 110 of the present disclosure and FIG. 1B shows schematics of how sensor-nanogenerator stents of the present disclosure can be used to continuously track local hemodynamic changes upon a re-narrowing condition. In the Patent Provisional Application No. 63/048,943 filed Jul. 7, 2020 (the “'943 Application,” incorporated by reference herein for all purposes), a meta-tribomaterial was presented with finely tailored and seamlessly integrated microstructures and composed of topologically different triboelectric materials for active sensing and energy harvesting. A preferred metamaterial cardiac stent 110 of the present disclosure comprises first and second conductive layers 112, 114 of a 2D MM with parallel semicircular-shaped snapping segments and a dielectric layer 116 as shown in FIG. 1A as built-in contact-electrification mechanisms for sensing and energy harvesting as disclosed in the '943 Application. In addition, the stents 110 of the present disclosure can be designed using layers of piezoelectric materials (lead zirconate titanate (Pb[Zr(x)Ti(1−x)]O3) (“PZT”) or polyvinylidene fluoride or polyvinylidene difluoride (“PVDF”)) to create a built-in piezoelectric nanogenerator for sensing and energy harvesting. For both triboelectric and piezoelectric nanogenerator modes, the structure of the metamaterial cardiac stents 110 is composed multi-stable/self-recovering segments. Their fabric is composed of finely tailored and topologically different auxetic microstructures (FIG. 1A). During cardiac perfusion, the stent structure undergoes periodic deformations due to the dilations of the artery. Deformation modes of the fabricated microstructures of metamaterial stents are designed in a specific way so that contact-electrification will occur between its triboelectric layers during each arterial pulsation. The contacting surfaces 112, 114 act as a conductive (electrode) and a non-conductive (dielectric) layer 116. As the stent expands during dilations, its snapping conductive layers 112, 114 and dielectric layer 116 separate from each other. No electric charge is generated during this phase. When the artery is relaxed (atrial diastole), additional forces caused by dilations are removed from the stent 110 and it automatically returns to its initial stable configuration because of its self-recovering snapping structure. In this phase, the conductive and dielectric layers, 112, 114 and 116, respectively, start accumulating positive and negative charges due to the contact electrification. When the structure of stent 110 is expanded in the next cardiac cycle, the transferred charge remains on the dielectric surface 116. This forms a static electric field and a potential difference between the conductive layers 112, 114, which results in generating an electrical output based on triboelectrification and electrostatic induction. In case of using piezoelectric materials as one of the layers in the lattice, the piezoelectric layer serves as a transducer to generate an electrical signal under each arterial pulsation.

The electrical output of stent 110 is proportional to the radial compressive forces applied to the structure of stent 110. Therefore, the electrical output signals can be used for active sensing of the radial force applied to the structure of stent 110, providing important information about the blood flow hemodynamics in the vessel. The stent signal can be used to characterize venous obstructions and vessel wall changes to assess deep vein thrombosis (DVT).

FIG. 1A shows first and second conductive layers 112, 114 of a 2D MM of stent 110 with parallel semicircular-shaped snapping segments 120 as well as dielectric layer 116 of stent 110.

Upon the onset of ISR when excess tissue or plaque builds up on the inner walls of the stent 110 to obstruct blood flow, hemodynamics and local pressure distribution around the stent 110 changes. Accordingly, the stent 110 radial force pattern shifts away from its baseline as an indication of ISR. The metamaterial stent 110 of the present disclosure is designed to function not only as a mechanical scaffold with tunable mechanical properties but also as both an energy harvesting transducer and a sensor directly collecting information about its operating environment without obstructing the blood flow. Note that stents 110 of the present disclosure naturally inherit the enhanced tunable mechanical properties offered by classical mechanical metamaterials such as ultra-high strength-to-density ratios and high resilience, which are crucial for a mechanically robust stent design. The preferred multi-layered meta-tribomaterial stents 110 of the present disclosure can be fabricated using a wide range of the biocompatible and bioresorbable materials.

In order to interrogate the metamaterial cardiac stents 110 wirelessly, this disclosure presents a novel non-invasive wireless communication approach using the built-in electric field of the cardiac stents 110. This preferred approach does not require any additional embedded electronics or inductive antennas to be integrated into the structures of stents 110. The bursts of electrical energy released by the composite lattice structure 122 of the metamaterial stents 110 during each cardiac cycle is significantly larger than the heart's electrical signal (0.5 mV to 1 mV range). The stent 110 uses Maxwell's displacement current principle for wireless transmission of the electrical signal. The strain-induced Maxwell's displacement current generated by the lattice structure 122 of metamaterial stent 110 enables wireless transmission of the sensed signal through various media without any antenna and power supply. The electrical output generated by the stent 110 has its distinct pattern in the ECG signal. The signal produced by stent 110 can be detected by electrodes placed on the skin and recorded by an electrocardiograph. The signal wave of stent 110 manifests as a tall wave in one of the chest (precordial) leads (e.g., in V1) (FIG. 1B). This interrogation approach can be used for an alternative wireless communication for any smart implant with built-in signal generation capabilities (e.g., piezoelectric-integrated implants).

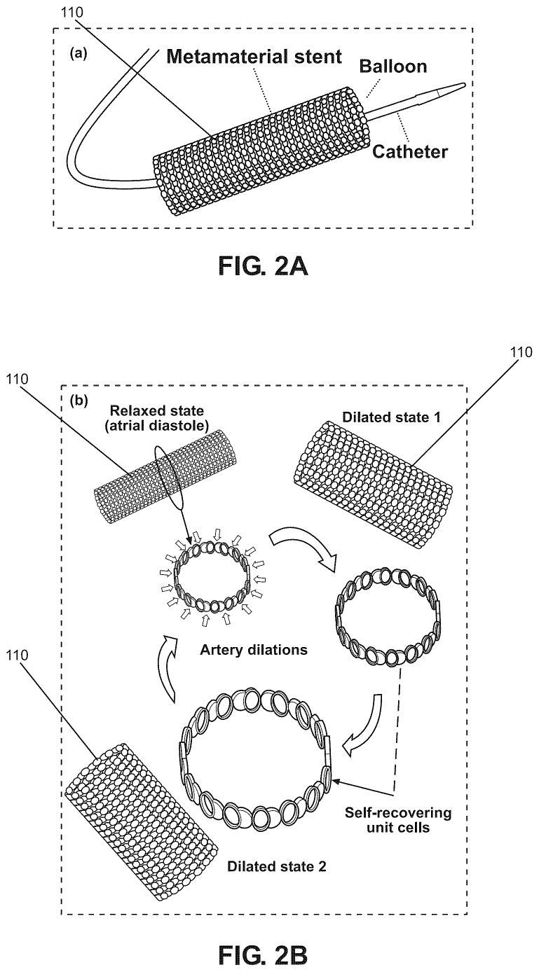

The preferred designs for the metamaterial stents 110 of the present disclosure with desired properties are schematically shown in FIG. 2A and FIG. 2B. Tailoring of the layers of the composite self-recovering metamaterial stents 110 (FIG. 2A and FIG. 2B) and their coupling response is critical for tuning the deformation and buckling shapes of the curve-shaped snapping elements 120 in the bi-stable unit cells of stents 110, and therefore for controlling the contact electrification between the triboelectric/piezoelectric layers. Preferably, stent 110 dilates and relaxes in response to arterial pulsations through self-recovering behavior. During these periodic deformations, contact electrification will occur between the rationally designed layers 112, 114 and 116 of stent 110. ISR changes local blood pressure and thus the response of the stent 110.

In addition to sensing and energy harvesting capabilities, another key feature of the metamaterial stents 110 is their tunable mechanical properties stemming from their composite lattice structure 122. At least two materials (one conductive (conductive layers 112, 114) and one non-conductive 116) with triboelectric properties are required in the design process of a metamaterial stent 110 to induce contact electrification. The composite lattice structure 122 can be made of metallic and/or polymeric materials. However, to introduce the self-recovering and flexibility behavior, at least one of the stent microlayers should be made from soft biocompatible materials with controllable flexibility (e.g. Thermoplastic Polyurethane (TPU): E=12 MPa; Polydimethylsiloxane, (PDMS or dimethicone): E=360 kPa-5 MPa). The other layers can be fabricated using stiffer biocompatible materials (e.g., stainless steel, platinum-chromium or cobalt-chromium alloys, Au, titanium, polylactic acid (PLA), polyetheretherketone (PEEK)). The mechanical tunability functionality is particularly important because of major concerns about the mismatch between the mechanical properties of the currently used stents and human blood vessels, which may impact the stenting performance and induce complications like vascular injury, thrombosis, dissection and more. Although polymeric stents exhibit a lower modulus (at least 100 times) comparted to metals, one of the major challenges associated with them is the risk of fracturing. The metamaterial stents 110 of the present disclosure are hybrid metamaterial systems that enable tackling these issues through their rational multi-layered design and choice of metallic and/or polymeric materials to improve radial strength, while they can still elongate, deform, and stretch under tensile stress during deployment without breaking the material. The auxetic microstructures of the metamaterial stents 110 along with their multi-stable/self-recovering and snapping behavior enable designing them with both self-expanding and balloon-expandable mechanisms. A balloon-expandable stent can be designed via strengthening the constrained conditions at both ends of the snapping curved segments 120 of each unit cell of the stent structure 110.

Furthermore, the metamaterial cardiac stents 110 of the present disclosure generate electrical signals that can be used for electrical stimulation to promote/accelerate wound healing (e.g., for critical limb ischemia (CLI) patients).

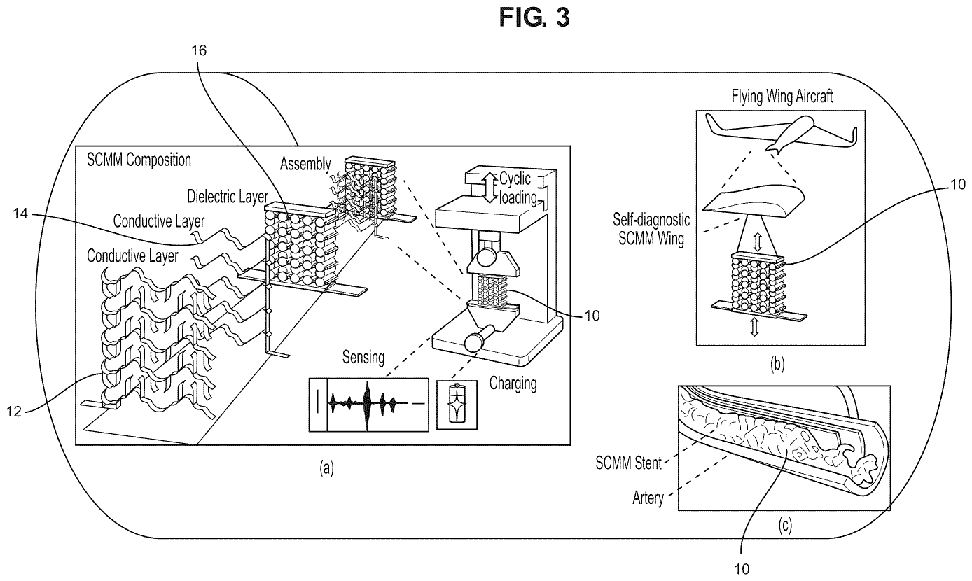

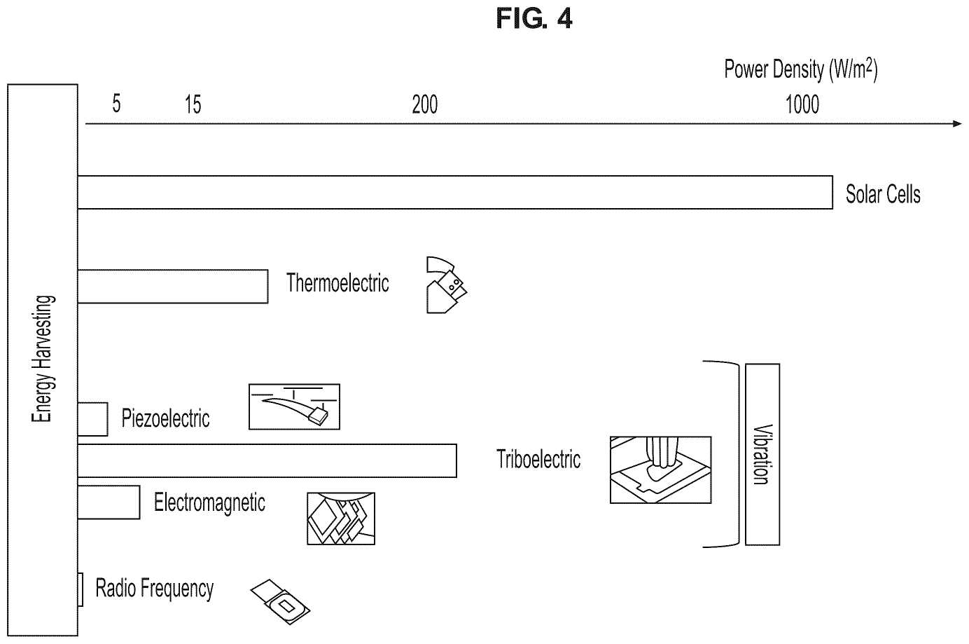

The present disclosure utilizes a new generation of composite MMs called “self-aware composite mechanical metamaterial (SCMM)” with complex internal structures toward achieving self-sensing and self-powering functionalities along with the boosted mechanical properties. The present disclosure is based on the following: (a) finely tailored and seamlessly integrated microstructures composed of topologically different triboelectric materials can form a hybrid MM system that can both harvest the energy from the external mechanical excitations and measure various levels of the forces applied to its structure; and (b) a composite MM composed of different materials that are organized in a periodic manner will boost the mechanical properties such as strength and stiffness. The feasibility of the SCMM concept of the present disclosure has been demonstrated for designing a metamaterial structure with active sensing and energy harvesting functionalities. The results have led to the grand vision for the present disclosure (shown in FIG. 3), where architecture tailoring of materials via additive manufacturing could form a new class of multifunctional MMs for a broad range of applications. FIG. 3 shows visions of the proposed multifunctional MM concept of the present disclosure for active sensing and energy harvesting: FIG. 3 shows in (a) a preferred composition of the SCMM system 10 of the present disclosure. FIG. 3 shows in (b) a flying wing aircraft with self-diagnostic and energy harvesting wings made of a network of SCMM structures 10. FIG. 3 shows in (c) a self-powered and self-sensing cardiovascular stent using SCMM 10 for continuous monitoring of the artery radial pressure changes due to tissue overgrowth. Deformation mode of the fabricated microstructures of the present disclosure preferably are engineered through a unique design so that contact electrification will occur between the two surfaces as the SCMM structure undergoes periodic deformations due to mechanical excitations. The SCMM contacting/sliding surfaces of the present disclosure will act as conductive and dielectric layers as shown in FIG. 3 at (a). Due to the contact electrification, the conductive and dielectric layers will accumulate positive and negative charges, respectively. As the SCMM structure of the present disclosure is unloaded, the transferred charge will remain on the dielectric surface. This will form a static electric field and a potential difference between the conductive layers. By increasing the loading amplitude, more conductive and dielectric layers of the SCMM matrix of the present disclosure will get involved in the contact-separation process, which will result in generating higher electrical output. The electrical output signals can be used for active sensing of the external mechanical excitation applied to the SCMM structure of the present disclosure. On the other hand, the generated electrical energy can be harvested and stored to empower sensors and electronics at low power.

The goal of the present disclosure is to advance the knowledge and technology required to create a new class of multifunctional MMs systems that offer self-sensing, self-monitoring, and energy harvesting properties along with boosted mechanical performance due to their composite structure.

The present disclosure preferably will aid in the discovery of materials with new properties and functionalities in the fields of aerospace (morphing/deployable space structures), biomedical devices (medical implants, stents, artificial muscles), civil infrastructure and construction. From a sensing perspective, introducing the self-sensing functionality into the MM design could in theory lay the foundations for living structures that respond to their environment and self-monitor their condition. The present disclosure preferably is directed to “self-aware structures” where structural systems utilize their entire constituent components as a sensing medium to directly infer multiple types of hidden information relating to the structure. In addition to its “inferring itself” aspects, the present disclosure has numerous applications in the structural health monitoring arena. Traditional structural health monitoring approaches use dedicated sensors which often results in dense and heterogeneous sensing systems that are difficult to install and maintain in large-scale structures. On the other hand, it is not always possible embed a traditional sensor (such as a strain gauge) inside structures such as, in which cross-sectional or interlaminate failures may not be observable at the surface. Another bottleneck limiting the structural health monitoring applications is that permanent monitoring systems often require extensive maintenance as a consequence of the limited durability of traditional sensors and of the limited robustness and exposure to failures of typical structural health monitoring architectures. The present disclosure can address most of these challenges because it is a paradigm shift in technology where structure can be a sensing medium itself through a rational architectural design and choice of constituent materials. In addition to its self-sensing features, an SCMM system of the present disclosure is intrinsically sensitive to the applied stresses, and therefore, it can be implemented as a sensor in various materials or structural systems.

From an energy harvesting perspective, the present disclosure offers new concepts and mechanisms for materials and structures that utilize the energy that develops within them (strain and kinetic energy) for self-powering or local powering of sensing and actuating devices.

From a mechanical perspective, SCMMs of the present disclosure are preferably composed of different materials that are organized in a periodic manner. Therefore, SCMMs of the present disclosure not only inherit all features of classical MMs but could also offer significantly boosted mechanical properties due to their composite structure by overcoming the “rule of mixtures”. In accordance with the present disclosure, mechanical properties of SCMMs are preferably predicted and tuned the to make them programmable tools for various engineering applications.

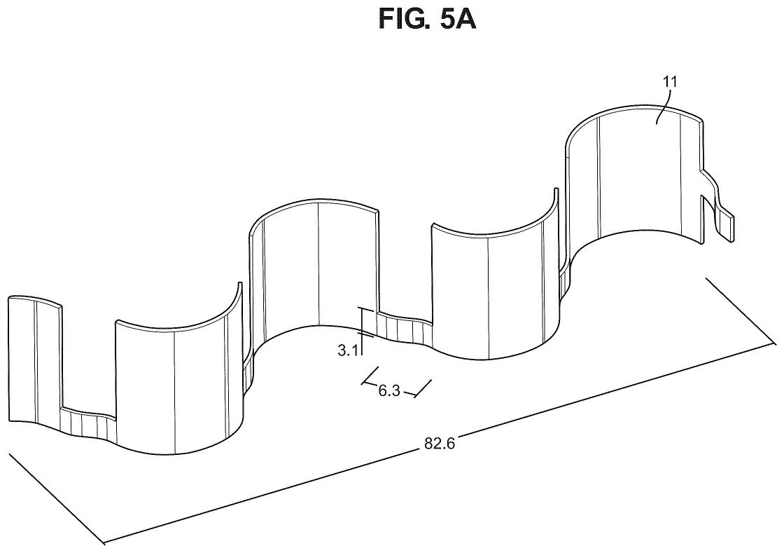

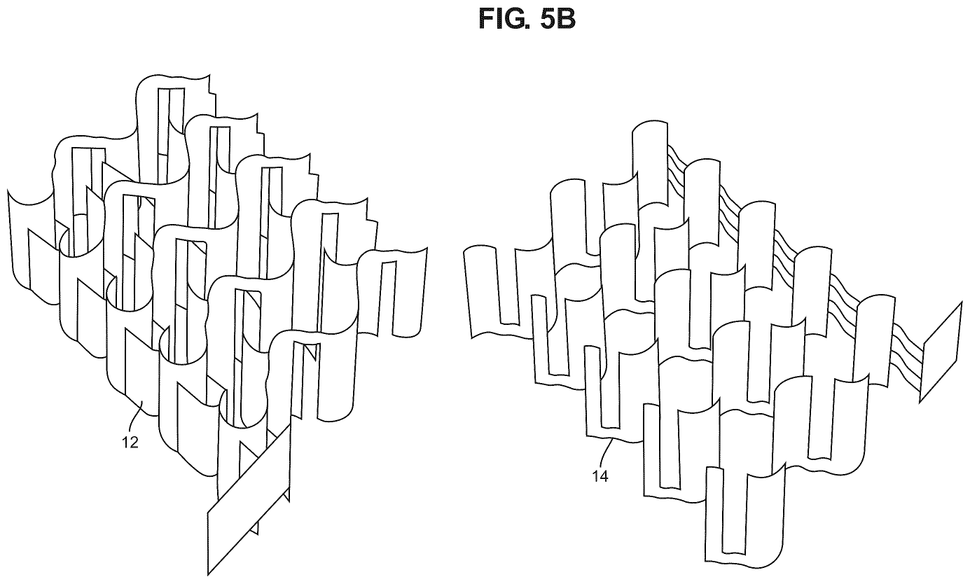

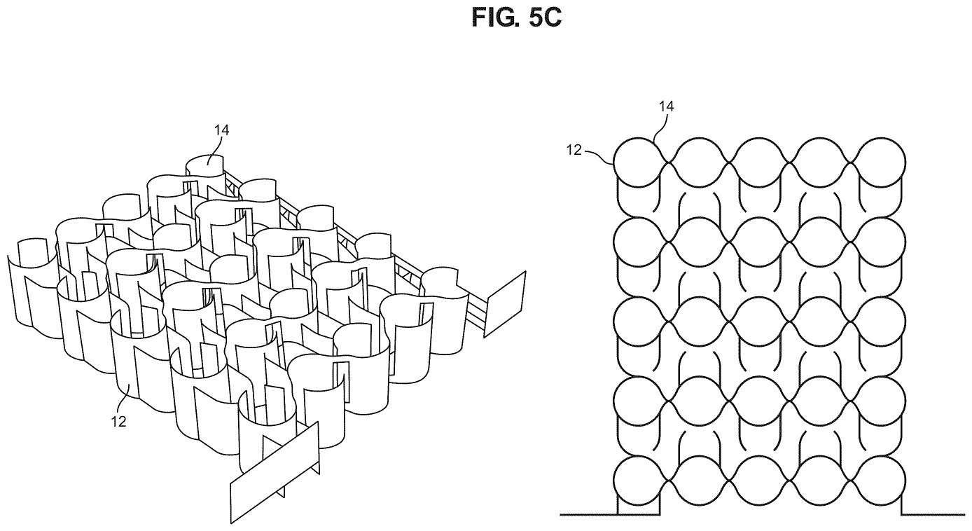

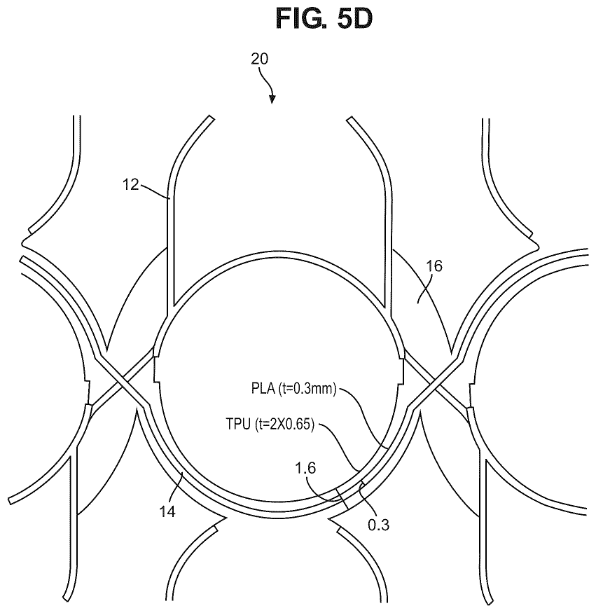

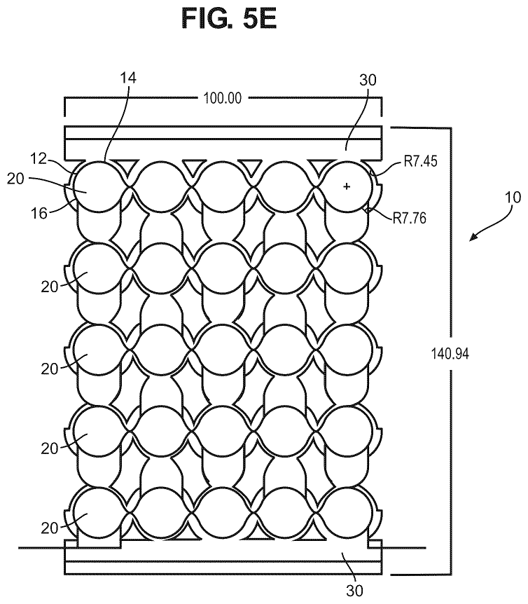

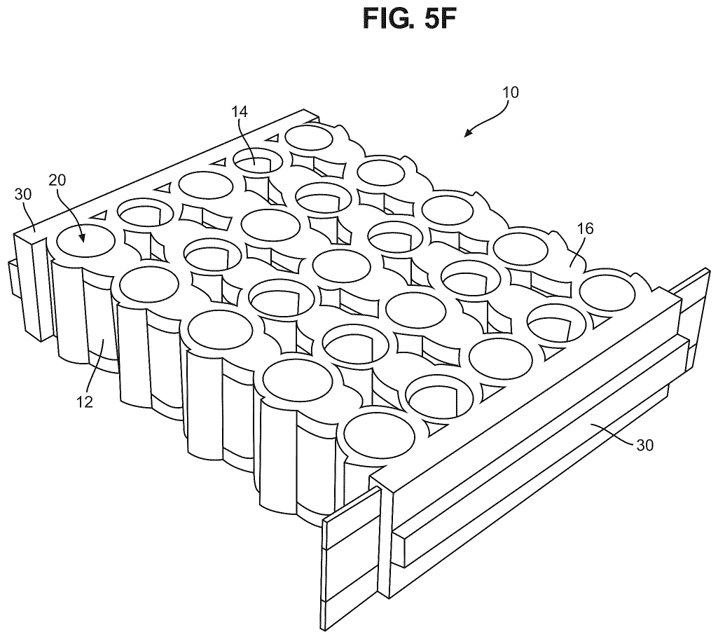

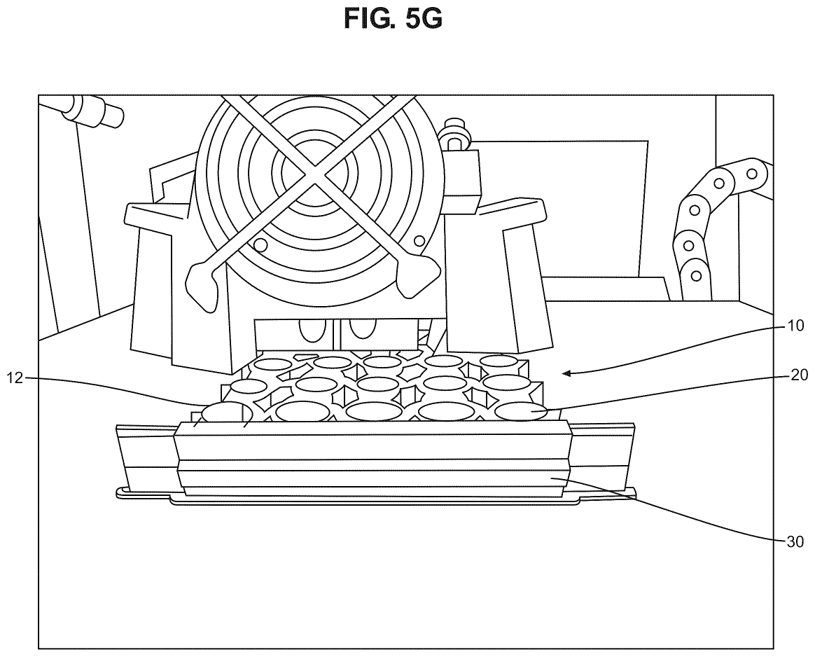

The performance of a two-dimensional (2D) snapping MMs 10 designed according to the SCMM concept of the present disclosure has been analyzed. An architected MM 10 containing parallel snapping curved (semicircular-shaped) segments 12, 14 with elastic snap-through instability mechanism was fabricated according to the present disclosure. The design of MMs with snap-through instabilities has been the focus of active research in recent years. Multi-stable/self-recovering snapping metamaterials have advantages in applications such as the development of tunable metamaterials with switchable properties. According to the present disclosure, the metamaterial was made up of multiple bi-stable unit cells 20. The unit cell 20 consisted of thick horizontal and vertical elements and a thin curved part. In order to incorporate the sensing and energy harvesting features into the metamaterial functionality of the SCMM 10 of the present disclosure, the triboelectrification phenomenon was incorporated into its architecture design. The triboelectrification phenomenon is a universally existing phenomenon in the nature and people's living life and has been known for thousands of years. It describes a contact electrification phenomenon that a material/surface becomes electrically charged after it gets into contact with a different material/surface. The design process is shown in FIGS. 5A-5G.

FIGS. 5A-5G show the designing of a 2D MM with parallel semicircular-shaped snapping segments according to the SCMM concept of the present disclosure. FIG. 5A shows a segment 11 of the conductive layers 12, 14 of a preferred 2D MM with parallel semicircular-shaped snapping segments of the present disclosure. FIG. 5B shows the two conductive layers 12, 14 created as 5 periodic repeatable segments. FIG. 5C shows aligned conductive layers 12, 14 of a preferred 2D MM with parallel semicircular-shaped snapping segments of the present disclosure. FIG. 5D shows the geometry of a symmetric unit cell 20 composed of the conductive layers 12, 14 and dielectric layers 16 that are involved in the contact-separation process of a preferred 2D MM with parallel semicircular-shaped snapping segments of the present disclosure. FIG. 5E and FIG. 5F show a schematic representation of the entire composite matrix of the SCCM 10 composed of the conductive layers 12, 14 and dielectric layers 16 in a periodic manner in a preferred 2D MM with parallel semicircular-shaped snapping segments of the present disclosure. FIG. 5G shows a preferred manner of 3D printing a SCCM 10 of the present disclosure.

In order to fabricate the 2D structure of the snapping SCMM of the present disclosure, three constituent layers were defined. The first two layers were conductive layers 12, 14 created as periodic repeatable segments 20 (FIGS. 5A-5B). The conductive layers 12, 14 were first aligned to act as opposite electrodes. Then, they were embedded inside a thicker dielectric layer 16 serving as the skeleton of the MM (FIG. 5D). As seen in FIGS. 5D-5F , the entire snapping SCMM structure 10 of the present disclosure forms composite matrix composed of the conductive layers 12, 14 and dielectric layers 16 in a periodic manner. The semicircular-shaped snapping segments include both conductive layers 12, 14 and dielectric layers 16 (FIG. 5D). The semicircular-shaped snapping segments 20 were centrally clamped by relatively thicker (stiffer) supporting segments 30 with a connection platform, as illustrated in FIGS. 5D-5E.

Preferably, the curved elements were specifically designed in mathematical/trigonometric function form to achieve smooth snap-through transition and symmetrical stable configurations before and after large deformation. In order to fabricate this complex design as one integrated unit, Raise3D Pro2 Dual Extruder 3D Printer was used as it supports printing with a variety of multi-material filaments. There is a wide range of organic and inorganic materials from the triboelectric series that can be used to fabricate the conductive and dielectric layers. Preferably, materials with a large difference in triboelectric polarity are used to maximize the electrification between the two layers. Polylactic Acid (PLA) with carbon black (Young's modulus E=3000 MPa, Poisson's ratio ν=0.48) and Thermoplastic Polyurethane (TPU) (E=12 MPa, ν=0.25) were used as the conductive and dielectric layers, respectively. The test setup and the fabricated SCMM of the present disclosure are shown in

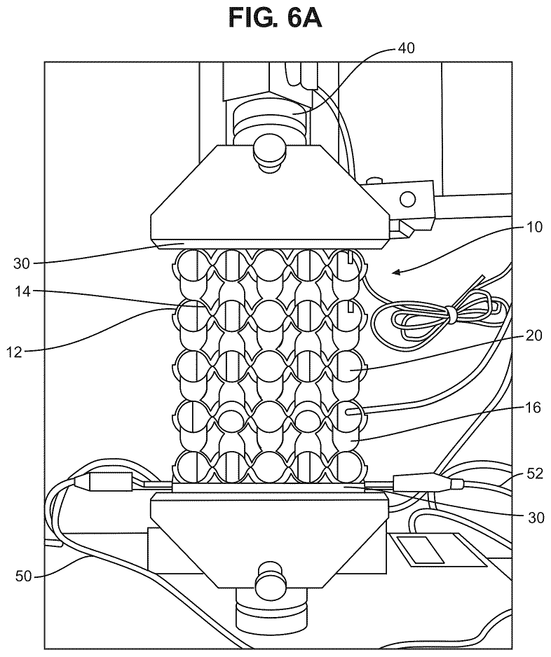

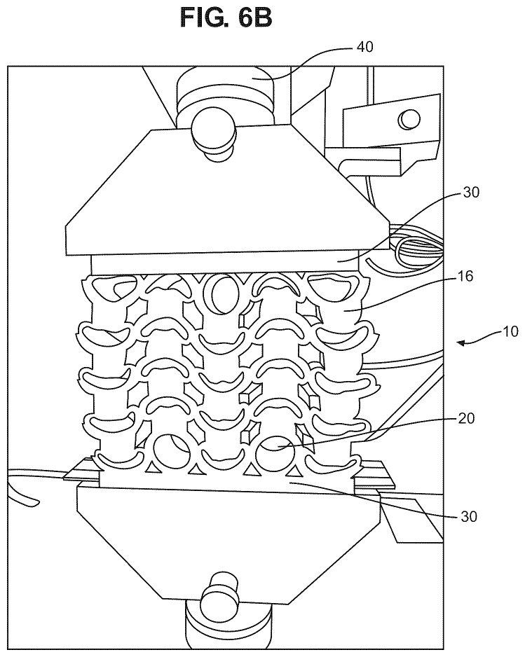

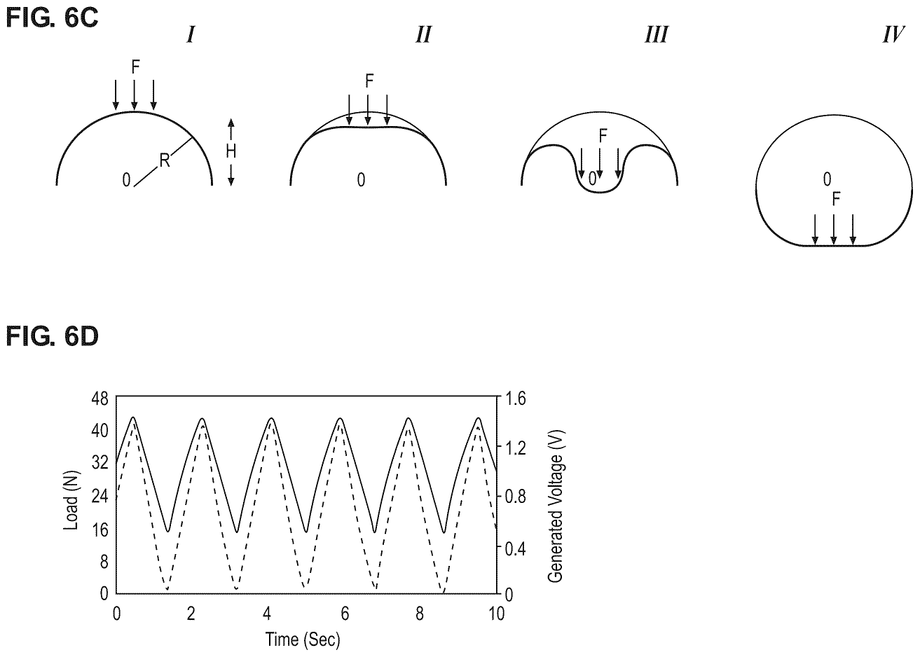

FIG. 5G and FIGS. 6A-6B . Uniaxial loading experiments were performed on the 3D printed metamaterial specimen SCCM 10 of the present disclosure with a TestResources testing machine 40. Cycling loading at 0.5 Hz frequency was applied to the specimen SCCM 10 under displacement control until it was fully compacted. The displacement range was controlled to be between 5 mm to 10 mm. The applied load changed between 15 N and 45 N. Under uniaxial loading, the SCCM 10 undergoes a large deformation caused by stiffness mismatch between snapping (buckling instabilities) and supporting (relatively stiffer/thicker) components, exhibiting very small transverse deformation after every snapping. Based on the multi-stable/self-recovering mechanism, phase transformation/shape-reconfiguration and zero (or close to zero) Poisson's ratio can be achieved up to large morphological change (FIG. 6B).

As shown in FIG. 6C, when a normal vertical force applied in the middle of the curved beams, the semicircular-shaped segment is mechanically deformed (buckled), snapping from first/original stable state (State I) to second/deformed stable state (State IV) at a critical applied force. In a very ideal situation, the constrained conditions at both ends are strong and the two stable states are symmetric, the reaction force will be symmetric from one to the other stable state under displacement control which means that an identical reverse force is needed that allows the deformed beams to return to their original configuration. In the case of a self-recovering snapping, the constant positive force means that the snapped segments (State IV) automatically return to their initial stable configuration (State I) after the load is removed. Under compressive loads, the SCCM 10 of the present disclosure undergoes periodic deformations and contact electrification occurs between the conductive layers 12, 14 and dielectric layers 16. By unloading the SCCM 10, a potential difference is formed between the conductive layers 12, 14. Higher loading amplitude results in larger deformations. Consequently, more conductive layers 12, 14 and dielectric layers 16 of the matrix of SCCM 10 get involved in the contact-separation process. This leads to higher rate of the electrostatically-induced electron transfer and generating higher voltage. In order to record the voltage generated due to the applied mechanical excitations, wires 50, 52 were connected to the extended parts of the conductive layers, as shown in FIG. 6A. The voltage values were read using a National Instruments 9220 DAQ module with 1 GΩ impedance. The applied cyclic loads and the corresponding voltage generated by the proposed mechanical metamaterial structure are shown in FIG. 6D. As seen, the voltage is proportional to the applied force. Also, the generated electrical energy can be readily stored using an energy harvesting circuit.

FIG. 6A shows a preferred self-sensing and self-charging 2D SCMM 10 of the present disclosure with a 5×5 array of unit cells 20. FIG. 6B shows the SCCM 10 in the compacted state. FIG. 6C shows preferred snapping mechanisms of elastic bulking semicircular-shaped snapping shells of the present disclosure. FIG. 6D shows applied cyclic loads and the corresponding voltage generated by the SCCM 10.

According to the present disclosure, it is feasible to create SCMMs 10 with sensing and energy harvesting functionalities via introducing the contact electrification into the fabrication process. Preferably, the SCMMs 10 of the present disclosure will allow for measuring the force applied to the SCMM 10 by monitoring the voltage generated therefrom. The kinetic energy harvested from the external excitations of the SCMM 10 can be stored for self-powering or empowering other sensing devices. Furthermore, the SCMMs 10 of the present disclosure allow for the creation of MMs whose mechanical and electrical response can be programmed. Preferably, the snapping mechanism or the layered design of the composite matrix of the SCMMs 10 of the present disclosure can be engineered to deform in specified order or prevent random snapping, which will result in programmed triboelectrification and mechanical behaviors. Preferably, the SCMMs 10 of the present disclosure can be applied to design a variety of programmable MMs with sensing, energy harvesting properties.

In the foregoing Detailed Description, various features are grouped together in a single embodiment to streamline the disclosure. This method of disclosure is not to be interpreted as reflecting an intention that the claimed embodiments of the disclosure require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A cardiovascular stent comprising:
an active mechanical metamaterial, comprising:
first and second electrically conductive components disposed relative to each other to act as opposite electrodes to induce contact electrification;
wherein the first and second electrically conductive components, along with a dielectric component serving as a skeleton of the active mechanical metamaterial, form a lattice of snapping curved semicircular-shaped segments, wherein each of the snapping curved semicircular-shaped segments has an elastic snap-through instability mechanism; and
wherein the lattice comprises periodic repeatable parallel rows of the snapping curved semicircular-shaped segments.

2. The cardiovascular stent of claim 1, wherein one of the first and second electrically conductive components comprises a piezoelectric material that serves as a transducer to generate an electrical signal under an arterial pulsation.

3. The cardiovascular stent of claim 1, wherein a signal produced by the cardiovascular stent can be detected by electrodes placed on a user's skin and recorded by an electrocardiograph.

4. The cardiovascular stent of claim 1, wherein each of the electrically conductive components and the dielectric component comprises one or more materials selected from the group of polyurethane, polydimethylsiloxane, stainless steel, platinum-chromium, cobalt-chromium alloys, gold, titanium, polylactic acid and polyetheretherketone.

5. The cardiovascular stent of claim 1, wherein a first one of the electrically conductive components comprises one or more materials selected from the group of polyurethane and polydimethylsiloxane; and wherein each of a second one of the electrically conductive components and the dielectric component comprises one or more materials selected from the group of stainless steel, platinum-chromium, cobalt-chromium alloys, gold, titanium, polylactic acid and polyetheretherketone.

6. The cardiovascular stent of claim 1, wherein one or more of the electrically conductive components and the dielectric component comprises one or more materials selected from the group of lead zirconate titanate, polyvinylidene fluoride or polyvinylidene difluoride.

7. The cardiovascular stent of claim 1, wherein one or more of the electrically conductive components and the dielectric component comprises one or more materials composed of auxetic microstructures.

8. The cardiovascular stent of claim 1, wherein the first and second electrically conductive components are embedded in the dielectric component.

9. The cardiovascular stent of claim 1, wherein a structure of the active mechanical metamaterial forms a composite matrix of the electrically conductive and dielectric components in a periodic manner.

10. The cardiovascular stent of claim 1, wherein each of the snapping curved semicircular-shaped segments comprises a portion of each of the first electrically conductive component, the second electrically conductive component and the dielectric component.

11. The cardiovascular stent of claim 1, wherein the lattice comprises a 5 by 5 array of the snapping curved semicircular-shaped segments.

12. The cardiovascular stent of claim 2, wherein the lattice comprises a 5 by 5 array of the snapping curved semicircular-shaped segments.

13. The cardiovascular stent of claim 3, wherein the lattice comprises a 5 by 5 array of the snapping curved semicircular-shaped segments.

14. The cardiovascular stent of claim 4, wherein the lattice comprises a 5 by 5 array of the snapping curved semicircular-shaped segments.

15. The cardiovascular stent of claim 5, wherein the lattice comprises a 5 by 5 array of the snapping curved semicircular-shaped segments.

16. The cardiovascular stent of claim 6, wherein the lattice comprises a 5 by 5 array of the snapping curved semicircular-shaped segments.

17. The cardiovascular stent of claim 7, wherein the lattice comprises a 5 by 5 array of the snapping curved semicircular-shaped segments.

18. The cardiovascular stent of claim 8, wherein the lattice comprises a 5 by 5 array of the snapping curved semicircular-shaped segments.

19. A cardiovascular stent comprising:
an active mechanical metamaterial, comprising:
first and second electrically conductive components disposed relative to each other to act as opposite electrodes to induce contact electrification;
wherein the first and second electrically conductive components, along with a dielectric component serving as a skeleton of the active mechanical metamaterial, form a lattice of snapping curved semicircular-shaped segments, wherein each of the snapping curved semicircular-shaped segments has an elastic snap-through instability mechanism;
wherein the lattice comprises periodic repeatable parallel rows of the snapping curved semicircular-shaped segments; and
wherein one of the first and second electrically conductive components comprises a piezoelectric material that serves as a transducer to generate an electrical signal under an arterial pulsation.

20. A cardiovascular stent comprising:
an active mechanical metamaterial, comprising:
first and second electrically conductive components disposed relative to each other to act as opposite electrodes to induce contact electrification;
wherein the first and second electrically conductive components, along with a dielectric component serving as a skeleton of the active mechanical metamaterial, form a lattice of snapping curved semicircular-shaped segments, wherein each of the snapping curved semicircular-shaped segments has an elastic snap-through instability mechanism;
wherein the lattice comprises periodic repeatable parallel rows of the snapping curved semicircular-shaped segments; and
wherein a first one of the electrically conductive components comprises one or more materials selected from the group of polyurethane and polydimethylsiloxane; and wherein each of a second one of the electrically conductive components and the dielectric component comprises one or more materials selected from the group of stainless steel, platinum-chromium, cobalt-chromium alloys, gold, titanium, polylactic acid and polyetheretherketone.
